(19) United States
(12) Patent Application Publication
(10) Pub. No.: US 2021/0113459 A1
(43) Pub. Date: Apr. 22, 2021

(54) CHEWABLE GEL FORMULATIONS

(71) Applicant: Santa Cruz Nutritionals, Santa Cruz, CA (US)

(72) Inventors: Scott Vogt, Aptos, CA (US); Justin Reyes, Salinas, CA (US); Booker Lucas, Danville, CA (US); Michael Simpson, Mooresville, NC (US)

(73) Assignee: Santa Cruz Nutritionals, Santa Cruz, CA (US)

(21) Appl. No.: 17/073,068

(22) Filed: Oct. 16, 2020

Related U.S. Application Data
(60) Provisional application No. 62/915,978, filed on Oct. 16, 2019.

(51) Int. Cl. A61K 9/00; A61K 9/06; A61K 47/36; A61K 47/26; A61K 31/519; A61K 36/28; A61K 33/06

(52) U.S. Cl. CPC A61K 9/0056; A61K 9/06; A61K 47/36; A61K 33/06; A61K 31/519; A61K 36/28; A61K 47/26

(57) ABSTRACT

Chewable gel formulations comprising agar, locust bean gum, starch, sugar, water, and about 15 wt. % to about 40 wt. % of vitamins, minerals or mixtures thereof, and related methods of use.

### RELATED APPLICATIONS

This application claims priority to U.S. provisional application No. 62/915,978, filed Oct. 16, 2019, the entirety of which is incorporated by reference.

### FIELD OF THE INVENTION

The present invention generally relates to chewable gel formulations and, more specifically, to chewable gel formulations that provide for the oral delivery of vitamins and/or minerals, as well as related methods for their administration.

### BACKGROUND

Chewable gel formulations are well known. While originated as a confection, these formulations have been adapted over the years to provide a means of delivering a variety of beneficial ingredients, such as active pharmaceutical ingredients and dietary supplements, as the chewable gels were found to have particular utility for persons who experience difficulty swallowing tablets and capsules.

Dietary supplements, which include, e.g., one or more of the following ingredients: vitamins, minerals, protein & amino acids, herbs or other botanical ingredients, have become increasingly popular over the past decade. As this market continues to grow, there exists a need for chewable gel formulations that provide enhanced properties relative to existing products.

### BRIEF SUMMARY OF THE INVENTION

The present invention, in one embodiment, provides chewable gel formulations comprising, consisting essentially of, or consisting of, about 0.1 to about 5 wt. % agar, about 0.01 to about 2 wt. % locust bean gum, about 0.1 to about 5 wt. % starch, about 30 to about 60 wt. % sugar, about 25 to about 50 wt. % carbohydrate syrup, and about 10 to about 30 wt. % water.

In a related embodiment, the present invention provides chewable gel formulations comprising, consisting essentially of, or comprising, agar, locust bean gum, starch, sugar, water, and about 15 wt. % to about 40 wt. % of vitamins, minerals or mixtures thereof.

In yet another related embodiment, the present invention provides chewable gel formulations comprising, consisting essentially of, or consisting of, about 0.1 to about 5 wt. % agar, about 0.01 to about 2 wt. % locust bean gum, about 0.1 to about 5 wt. % starch, about 30 to about 60 wt. % sugar, about 25 to about 50 wt. % carbohydrate syrup, about 10 to about 30 wt. % water, and about 15 wt. % to about 40 wt. % vitamins, minerals or mixtures thereof.

Methods of administering the various embodiments of the chewable gel formulations described herein are also contemplated by the present invention.

### DETAILED DESCRIPTION OF THE INVENTION

The present invention provides chewable gel formulations, and related methods of administration, that may be used as a carrier for relatively high concentrations of vitamins, minerals and mixtures thereof, while further avoiding undesirable properties associated with known chewable gel formulations. In related embodiments, the invention provides relatively high concentrations of vitamins, minerals and mixtures thereof in the aforesaid carriers. Illustrative of the properties exhibited by the inventive formulations include, for example, and desirably after storage under certain conditions, one or more of low or no syneresis and minimal self-adhesion without sanding, as well as minimal tailing (during manufacture) and acceptable texture, firmness, moisture content, water activity, bite, stability and mouthfeel.

The inventive formulations provide these and other advantages via the combination of agar, locust bean gum, starch, sugar and water, with certain embodiments also including relatively high concentrations of vitamins, minerals or mixtures thereof. That the formulations which include relatively high-loading of vitamins and/or minerals provide various additional benefits relative to existing chewable products which include vitamins and/or minerals, including, for example, permitting the delivery of the same (or greater) amount of vitamins and/or minerals to a subject with less sugar and lower mass (as assessed on a per serving basis), as well as other advantages as described herein.

In addition, and in yet another embodiment, the formulations desirably are free of other gel-forming hydrocolloids such as, e.g., pectins, alginate, gelatin, gellan, celluloses, and carrageenan.

In further embodiments, the inventive formulations desirably forego sanding, a process in which a sugar or other desiccant is applied onto the outer surface of the gel formulation, commonly used as a means of controlling undesirable syneresis and self-adhesion. More specifically, the formulations, when their production is completed and they are within a package for commercial distribution, have no water absorbent excipient or desiccant on their outer surface. In other words, the outer surface of the pieces, in the form in which they will be packaged and marketed commercially, have not been subjected to post-production (after shakeout) sanding, and are thus free of a water-absorbent excipient or desiccant.

In other embodiments, the inventive formulations provide the desirable properties described herein in the absence of ingredients comprising divalent cations, such as salts of calcium and magnesium.

The inventive formulations comprise a gel structure which is provided primarily via the combination of agar, locust bean gum (LBG) and starch, and desirably in certain amounts. It is believed that this combination is at least partially responsible for the desirable formulation properties described herein, which are provided despite the inclusion of relatively high concentrations (about 15 wt. % to about 40 wt. %) of vitamins, minerals or mixtures thereof.

Agar (also referred to as agar-agar) is a hydrocolloid, and more specifically a thermoreversible gelling agent comprised of two polysaccharides, agarose and agaropectin, with agarose being the primary gel-forming component. Agar is soluble in water at relatively higher temperatures, e.g., in excess of 80° F. and more typically in excess of 100° F., is compatible in the inventive formulations despite the relatively high amount of sugars, and is believed to assist in imparting temperature stability and a reduction in self-adhesion to the formulations.

Agar generally may be present in the formulations in an amount ranging from about 0.01 wt. % to about 10 wt. %, desirably from about 0.05 wt. % to about 8 wt. %, more desirably from about 0.1 wt. % to about 5 wt. %, even more desirably from about 0.1 wt. % to about 2 wt. %. Preferably, agar is present in the formulations in relatively lower amounts, ranging from about 0.1, 0.2, 0.3, 0.4, 0.5, 0.6, 0.7, 0.8 or 0.9 wt. % up to about 1.0, 1.1, 1.2, 1.3, 1.4 or 1.5 wt. %, with amounts ranging from about 0.3 wt. % to about 1.4 wt. %, about 0.5 wt. % to about 1.3 wt. %, and about 0.8 wt. % to about 1.2 wt. % being more preferred, and about 0.9 wt. % to about 1.1 wt. % begin even more preferred.

Locust bean gum (LBG), a water-soluble vegetable gum extracted from the seed of the carob tree, is a hydrocolloid comprised primarily of relatively high molecular weight hydrocolloidal polysaccharides. LBG is believed to contribute to the increase in viscosity (thickening) of the formulation, however, it is further believed that LBG has no, or at best a minor, role in gel formation. LBG also is sensitive to relatively high and low pH and relatively high temperatures, wherein it can degrade and lose its viscosity-enhancing properties.

LBG may be included in the formulations of the invention in any suitable amount depending primarily on the desired viscosity of the formulation during processing, but is desirably included at from about 0.001 wt. % to about 1 wt. %, more desirably from about 0.01 wt. % to about 0.5 wt. %, and even more desirably from about 0.01 wt. % to about 0.2 wt. %. Preferably, LBG may be present in the formulations ranging from about 0.04, 0.05, 0.06, or 0.07 wt. % to about 0.08, 0.09, 0.1, 0.11, 0.12, 0.13, 0.15, 0.17 or 0.2 wt. %, with amounts ranging from about 0.4 wt. % to about 0.15 wt. %, about 0.5 wt. % to about 0.12 wt. %, and about 0.6 wt. % to about 0.10 wt. %, being more preferred, and about 0.7 wt. % to about 0.9 wt. %, as well as at about 0.1 wt. %, being most preferred.

Starch is also a component of the formulations. The starch assists in increasing the viscosity of the formulation as it is heated during processing, but it is believed that the starch also may stabilize the water within the formulation, enhance gel formation and strength, permit less sugar to be used, increase the softness, decrease the firmness, and decrease the toughness, of the formulations, and also assist in reducing syneresis and self-adhesion (tackiness) of the formulations—despite the formulations permitting relatively high loading of vitamins and/or minerals, as well as in other embodiments in which ingredients containing one or more divalent minerals (e.g., calcium and magnesium) are omitted. In certain formulations, it was observed that the self-adhesion, or tackiness, was relatively the same after shakeout (between about 2-8 hours after shakeout, and within the same day of production) and two days after shakeout, this providing advantages in processing, e.g., the product is able to be packaged after shakeout without waiting 24-48 hours for the tackiness to be reduced, and the product may be commercialized without sanding.

While a variety of starches may be used, thin boiling (and acid thinned) starches, commonly prepared via treating starch or starch granules (e.g., corn, wheat, potato and tapioca starch) with inorganic acids to reduce their viscosity in water, and native starches, are desired. In particular, those starches that fracture at relatively low temperatures are more preferred e.g., desirably below about 130° C., and more desirably between about 110° C. and 130° C. While fracturing, followed by gelatinization, of the starch is desired during preparation of the formulations, it is desirable to avoid total, or complete, fracturing of the starch because this contributes to undesirable properties in the finished formulation, e.g., lumpiness.

Even more preferred are thin boiling and native starches having a Water Fluidity of between about 50-80 WF, and more preferably between about 60-75 WF, as starches with these WF values balance low viscosity when heated with thickening strength—the lower the number the higher the hot viscosity but the lower the resulting gel strength in the finished formulation. Starches further having relatively low amylose (e.g., between about 5% to about 40% amylose) also are desirable, as starches having relatively high amylose levels (e.g., above about 55%) typically require higher cooking temperatures, and also set too fast, the latter contributing to undesirable tailing.

Starch may be included in the formulations of the invention in any amount that assists in providing one or more of the desirable properties exhibited during processing or in the finished formulation, but is desirably included at from about 0.01 wt. % to about 10 wt. %, more desirably from about 0.1 wt. % to about 7 wt. %, even more desirably from about 0.5 wt. % to about 5 wt. %, and most desirably from about 1 wt. % to about 3 wt. % and even more desirably from about 1 wt. % to about 2 wt. %. Preferably, the starch may be present in the formulation from about 0.1, 0.2, 0.3, 0.4, 0.5, 0.6, 0.7, 0.8 or 0.9 wt. % to about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0 wt. %, with amounts ranging from about 0.5 wt. % to about 2.5 wt. %, about 0.7 to about 2 wt. % being more preferred, and about and 0.8 wt. % to about 1.7 wt. % being most preferred.

It was determined that the weight ratios of these three ingredients also assisted in imparting desirable attributes to the inventive gel formulations, while also avoiding undesirable properties. For example, the weight ratio of agar to LBG in the formulations may vary, but is desirably controlled so as to range from about 40:1 to about 1:1, more desirably from about 30:1 to about 5:1, even more desirably from about 25:1 to about 10:1, and preferably from about 20:1 to about 10:1. Similarly, the agar to starch weight ratio may vary, but desirably ranges from about 5-20:10-30, more desirably from about 7-20:15-25, and more desirably about 10-15:20.

The weight ratios of all three components, agar, LBG and starch, also desirably may be maintained at from about 40-1:1:40-1, more desirably from about 30-10:1:30-10, even more desirably from about 20-10:1:30-10, and preferably from about 15-10:1:25-15.

The formulations further include sugar, desirably in an amount ranging from about 20 wt. % to about 60 wt. %, and desirably ranging from about 25 wt. % to about 50 wt. %.

The formulations also include a carbohydrate syrup. The syrup may be selected from one or more of a variety of such syrups, but are desirably one or more of corn syrup, tapioca syrup, rice syrup, pea syrup, glucose syrup or mixtures thereof. The syrup may be used in any desired amount, but preferably is present in the formulation from about 20 wt. % to about 50 wt. %, and desirably from about 25 wt. % to about 40 wt. %.

Another formulation ingredient is water, which is believed to perform a plurality of functions, including assisting in gel formation and contributing to the desirable texture of the formulation. While water in any amount necessary to provide the desired properties in the formulation may be used, is it desirable that water be present in the formulation an amount ranging from about 5 wt. % to about 30 wt. %, more desirably from about 10 wt. % to about 20, and preferably from about 10 wt. % to about 15 wt. %.

The formulation also contemplates the inclusion of one or more of a vitamin, mineral, or mixtures thereof at relatively high concentrations ranging from about 15 wt. % to about 40 wt. %, including about 15 wt. %, about 16 wt. %, or about 18 wt. % to about 20 wt. %, about 25 wt. %, 30 wt. % or about 40 wt. %, the concentrations including each individual integer within these ranges. The vitamins and minerals that may be included in the inventive formulations are commercially available in various forms, for example, as vitamins and minerals per se, with some also available as salts, chelates and complexes. Recognizing this variety of forms, the amount (wt. %) of vitamins and/or minerals in the context of the present invention refers to the weight percent of the actual vitamin- or mineral-containing ingredient incorporated into the formulation. For example, if a mineral is included in its salt form, the weight percent of that mineral should be calculated based on the weight of the mineral salt. Similarly, if the mineral- or vitamin-containing ingredient is included as a chelate or complex, the weight percent of that ingredient should be calculated based on the weight of the chelate or complex.

In certain embodiments, the formulations include only minerals, such as calcium, magnesium, and in certain embodiments include only magnesium and/or its salts. In other embodiments, minerals constituting divalent cations, such, e.g., calcium and/or magnesium, may be omitted. In embodiments wherein the formulations include only minerals, or a mixture of minerals, vitamins and/or other optional ingredients, the amount of minerals that may be included in the formulations can be substantial while retaining one, or preferably more than one, of the desirable properties of the formulations described herein, e.g., no or minimal syneresis. In these embodiments, the mineral concentrations in the formulations may range from about 15 to about 40 wt. %, including about 15, about 20 or about 25 wt. % to about 30, about 35 or about 40 wt. %, the concentrations including each individual integer within these ranges.

Illustrative of minerals that may be included in the formulations are calcium (e.g., calcium carbonate or tricalcium phosphate), chloride (e.g., potassium chloride), chromium (e.g., chromium picolinate) copper (e.g., cupric oxide) iron (e.g., ferrous fumarate), zinc (e.g., zinc oxide), magnesium (e.g., magnesium oxide, magnesium citrate), iodine (potassium iodide), zinc (e.g., zinc citrate), selenium (e.g., selenium selenite), manganese (e.g., manganese citrate), chromium (e.g., chromium picolinate), molybdenum (e.g., molybdenum citrate), cobalt (e.g., cobalt gluconate), phosphorus (e.g., mono- or tri-calcium phosphate), potassium (e.g., potassium chloride), fluoride (e.g., sodium fluoride) and sodium (e.g., sodium chloride).

Any vitamin may be included in the inventive formulations, with each vitamin being included in any desired amount. Illustrative of vitamins contemplated by the inventive formulations are Vitamin A, Vitamin B1 (thiamine), Vitamin B2 (riboflavin), Vitamin B3 (niacin), Vitamin B5 (pantothenic acid), Vitamin B6 (pyridoxine), Vitamin B7 (biotin), Vitamin B9 (folate), Vitamin B12, Vitamin C, Vitamin D, Vitamin E, Vitamin K and chelates thereof.

One or more additional supplements also may be included in the formulations, e.g., antioxidants (e.g., lutein, lycopene, cinnamon, tumeric), folic acid, echinacea, garlic, fructans (e.g., inulin), herbs, or herbal extracts, but are not required.

The formulations may include other optional ingredients, such as colorants and flavorants, as are known in the art to be GRAS for use in consumables.

As mentioned previously, one advantage of the inventive formulations is that they provide for relatively high concentrations of vitamins, minerals and mixtures thereof, while exhibiting limited syneresis and self-adhesion (i.e., individual pieces of the formulation adhering to one another), also referred to as tackiness, post-production without sanding.

In an apparent effort to reduce syneresis and self-adhesion, currently available products incorporate sanding, which is the application of one or more of a desiccant, water-absorbent ingredient (e.g., a starch), or sugar (or sugar-substitute) on the exterior of the product post-shakeout. Embodiments of the present invention do not require sanding on the exterior surface of the formulation. The formulations instead may, if desired, be polished with a suitable GRAS ingredient, as is known in the art.

In addition, certain embodiments of the inventive formulations desirably exhibit, even after storage under certain conditions (e.g., for two weeks, one month, two months and/or three months at 25° C./35% RH), low or no syneresis and minimal self-adhesion without sanding, as well as minimal tailing (during manufacture) and acceptable texture, firmness, moisture content, water activity, bite, stability and mouthfeel.

Tackiness, or self-adhesion, of a finished formulation sample may be analyzed using a texture analyzer instrument (Stable Microsystems TA.XT Plus) onto which a TA-25 2 inch diameter aluminum 20 mm tall compression probe has been fitted. The analysis is completed while the instrument probe and the sample to be tested are at room temperature (23° C.±5° C.). The analysis begins by securing the sample to a workstation so that a non-starch contact portion of the sample is oriented so as to receive the probe, applying 100 g of force onto the sample via the probe for 10 seconds, followed by retraction of the probe from the sample. Upon retraction, the load cell instrument measures the negative force in grams, i.e., the force required to separate the probe from the sample as the probe is retracted. The measurement of tackiness is the force in grams required to separate the probe from the sample.

The tackiness of each formulation, as set for the herein is a numerical average obtained from the individual testing of 15 randomly selected samples.

One desirable aspect of certain of the inventive formulations is that the average tackiness of the formulation after shakeout (in grams) is substantially the same as the average tackiness two days after shakeout. In this regard, the average tackiness after shakeout (i.e., between about 2-8 hours after shakeout, and within the same day of production) is desirably no more than about 20%, more desirably no more than about 15%, even more desirably no more than about 10%, and preferably no more than about 5%, relative to the average tackiness of samples from the same batch assessed two days after shakeout (the samples having been stored in a closed container at (23° C.±5° C.)). Formulations having this property provide desirable manufacturing efficiencies, as the product may be packaged after shakeout (i.e., between about 2-8 hours after shakeout, and within the same day of production) without tackiness interfering with the packaging process or the desired product properties (e.g., pieces adhering to one another in the packaging).

On an absolute basis, the tackiness of a formulation (based on a numerical average of 15 sample pieces) after shakeout (between about 2-8 hours after shakeout and within the same day of production), and even more desirably after 2 days post-shakeout, desirably ranges from about 0.5 g to about 8 g, more desirably from about 1 g to about 7 g, and most desirably from about 2 g to about 6 g, preferably with a standard deviation (SD) of no more than about 1.5, more preferably no more than about 1.2, and even more preferably no more than about 1.

The firmness and toughness of formulation samples also may be assessed, desirably by testing a number of samples (e.g., 15) and determining a numerical average value for those samples. The same instrument used to assess tackiness (Stable Microsystems TA.XT Plus) fitted with a TA-54 4 mm probe is used to assess these two properties. The analysis is completed while the instrument probe and the sample to be tested are at room temperature (23° C.±5° C.). The analysis begins by securing the non-starch contact side of the sample onto a workstation, so that the probe contacts the side of the sample that has contacted the starch during production. Thereafter, the probe penetrates the sample to a depth of 10 mm at a speed setting of 1.0 mm/second. The instrument measures the peak force (in grams) required to penetrate the sample to a depth of 10 mm—this is the sample firmness. Sample toughness is the work (i.e., energy required) to insert the probe into the sample to a depth of 10 mm, and is reported in units of grams·sec.

On an absolute basis, the firmness of a formulation (based on a numerical average of 15 sample pieces) after shakeout (between about 2-8 hours after shakeout and within the same day of production), and even more desirably after 2 days post-shakeout, as tested in accordance with the firmness protocol described herein, desirably ranges from about 500 g to about 2000 g, more desirably from about 600 g to about 1500 g, and even more desirably from about 700 g to about 1000 g.

The toughness of a formulation, on an absolute basis, based on a numerical average of 15 sample pieces) after shakeout (between about 2-8 hours after shakeout and within the same day of production), and even more desirably after 2 days post-shakeout, as tested in accordance with the toughness protocol described herein, desirably ranges from about 1000 to about 15,000 grams·sec, more desirably from about 2500 to about 10,000 grams·sec, and even more desirably from about 3500 to about 7500 grams·sec, and preferably from about 4000 to about 6000 grams·sec.

The water activity of the formulations is desirably less than 0.75, and more desirably between about 0.01 and about 0.75, about 0.7, about 0.65 or about 0.6. Various methods and apparatus exist for determining water activity, and any may be used for purposes of assessing water activity.

The inventive formulations may be prepared in any conventional manner, including as a batch or continuous process. Generally, however, the formulation may be prepared via a batch process, desirably by hydrating agar and LBG in water to form an initial mixture, and incorporating sugar, carbohydrate syrup and starch (dispersed in water) therein. The resulting mixture is then heated, with any heat-resistant vitamins and minerals added thereto, if desired. After the mixture is boiled, an aqueous acidic solution may be added to lower the pH. Thereafter, the mixture is cooled to initiate formation of the gel structure, deposited into a plurality of molds, and allowed to fully cool.

The formulations as described herein provide for ease of production of individual molded products, as little or no undesirable tailing is observed.

By way of illustration only, the following tables provide a description of ingredients and amounts thereof that may be used to prepare formulations contemplated by the invention, and which may be used in the methods described herein, e.g., oral administration to supplement the diet of an individual requiring vitamin and/or mineral supplementation.

Table A describes ingredients and amounts (based on the wt. % of the ingredient in the finished formulation) of formulations useful as base formulations, permitting vitamins, minerals or mixtures thereof to be added thereto, as well as colorants and/or flavorings.

TABLE A

| Ingredient* | Formulation 1 | Formulation 2 | Formulation 3 |
|---|---|---|---|
| Agar | 0.5-2 | 0.8-1.6 | 1-1.3 |
| Locust Bean Gum | 0.05-2 | 0.07-1.5 | 0.09-0.12 |
| Starch | 0.1-5 | 0.5-2.5 | 1-1.5 |
| Carbohydrate Syrup | 25-60 | 30-55 | 35-45 |
| Sugar | 25-60 | 30-55 | 35-45 |
| Citric Acid** | 0.05-1 | 0.1-0.7 | 0.15-0.25 |
| Water | 10-20 | 10-15 | 11-14 |
| Polish | 0.05-1 | 0.05 0.5 | 0.1-0.15 |

*The amount of each ingredient identified in this example (other than the acidic ingredient which is already variable) may be varied ±10 wt. %.
**Amount may vary as needed to obtain desired pH.

Table B describes ingredients and amounts thereof (based on the wt. % of the ingredient in the finished formulation) of formulations which include relatively high levels of vitamins therein.

TABLE B

| Ingredient* | Formulation 4 | Formulation 5 | Formulation 6 |
|---|---|---|---|
| Agar | 0.5-2 | 0.8-1.6 | 0.9-1.1 |
| Locust Bean Gum | 0.05-2 | 0.06-1.5 | 0.07-0.1 |
| Starch | 0.1-5 | 0.5-3 | 1-2 |
| Carbohydrate Syrup | 20-50 | 25-45 | 30-40 |
| Sugar | 20-50 | 25-45 | 30-40 |
| Vitamin Mixture | 10-40 | 12-35 | 15-30 |
| Citric Acid** | 0.05-1 | 0.1-0.7 | 0.15-0.25 |
| Water | 10-20 | 10-15 | 10-15 |
| Polish | 0.05-1 | 0.05-0.5 | 0.1-0.15 |

*The amount of each ingredient identified in this example (other than the acidic ingredient which is already variable) may be varied ±10 wt. %.
**Amount may vary as needed to obtain desired pH.

Colorants and/or flavorings may be added to the formulations as desired.

Table C describes ingredients and amounts thereof that provide formulations which include relatively high levels of vitamins and calcium therein.

TABLE C

| Ingredient* | Formula 7 | Formula 8 | Formula 9 |
|---|---|---|---|
| Agar | 0.5-2 | 0.7-1.5 | 0.8-1.1 |
| Locust Bean Gum | 0.01-2 | 0.05-1.5 | 0.05-0.1 |
| Starch | 0.1-5 | 0.5-2 | 0.7-1 |
| Carbohydrate Syrup | 20-55 | 25-50 | 30-40 |
| Sugar | 20-55 | 25-50 | 30-40 |
| Tricalcium Phosphate | 5-30 | 7-25 | 10-20 |
| Vitamin Mixture | 5-20 | 5-15 | 5-10 |
| Citric Acid** | 0.05-1 | 0.1-0.5 | 0.1-0.3 |
| Water | 10-20 | 10-15 | 10-15 |
| Polish | 0.05-1 | 0.05-0.5 | 0.1-0.15 |

*The amount of each ingredient identified in this example (other than the acidic ingredient which is already variable) may be varied ±10 wt. %.
**Amount may vary as needed to obtain desired pH.

Colorants and/or flavorings may be added to the formulations as desired.

Table D describes ingredients and amounts thereof that provide formulations which include relatively high levels of magnesium therein.

TABLE D

| Ingredient | Formula 10 | Formula 11 | Formula 12 |
|---|---|---|---|
| Agar | 0.5-2 | 0.7-1.5 | 0.8-1.2 |
| Locust Bean Gum | 0.005-1 | 0.01-0.15 | 0.01-0.05 |
| Starch | 0.1-5 | 0.5-2.5 | 0.5-1.5 |
| Carbohydrate Syrup | 20-55 | 25-50 | 30-40 |
| Sugar | 15-40 | 20-35 | 20-30 |
| Magnesium Citrate | 10-45 | 15-40 | 20-35 |
| Citric Acid | 0.1-3 | 0.5-2 | 1-1.5 |
| Water | 1-15 | 2-10 | 3-7 |
| Polish | 0.05-1 | 0.05-0.5 | 0.1-0.15 |

*The amount of each ingredient identified in this example (other than the acidic ingredient which is already variable) may be varied ±10 wt. %.
** Amount may vary as needed to obtain desired pH.

Colorants and/or flavorings may be added to the formulations as desired.

### Example 1

A series of gummy base samples were prepared in an effort to assist in understanding the effect of varying amounts of three gelation agents (agar, locust beam gum (LBG) and starch (Ingredion Flojel® G (05060002) modified starch)) on the texture and tackiness of the finished samples. The samples did not include any vitamins or minerals, but were otherwise prepared using the same ingredients and method of preparation. Samples from each formulation batch (ID, total batch yield, in grams) were randomly selected for testing. The samples initially were tested between about two and eight hours after shakeout and within the same day of production, while the samples were at a temperature of 24° C.

The test for tackiness was undertaken as described in the specification hereinabove. The results from the tackiness test are provided in Table 1.

TABLE 1

| | Yield | Agar | Agar | LBG | LBG | Starch | Starch | Tack |
|---|---|---|---|---|---|---|---|---|
| ID | (g) | (g) | (wt. %) | (g) | (wt. %) | (g) | (wt. %) | (g) |
| 1 | 113.45 | 1.94 | 1.7 | 0.15 | 0.1 | 11 | 9.7 | 0.745 |
| 1a | 111.87 | 0.65 | 0.6 | 0.15 | 0.1 | 11 | 9.8 | 1.032 |
| 1b | 111.7 | 0.65 | 0.6 | 0 | 0 | 11 | 9.8 | 0.932 |
| 1c | 111.11 | 1.94 | 1.7 | 0.15 | 0.1 | 0 | 0 | 3.958 |
| 1d | 113.27 | 1.94 | 1.7 | 0 | 0 | 11 | 9.7 | 1.66 |
| 1e | 111.43 | 1.29 | 1.2 | 0.1 | 0.1 | 5.5 | 4.9 | 1.722 |
| 1f | 110.93 | 1.94 | 1.7 | 0 | 0 | 0 | 0 | 0.897 |
| 1g | 109.53 | 0.64 | 0.6 | 0.15 | 0.1 | 0 | 0 | 2.978 |
| 1h | 019.35 | 0.64 | 0.6 | 0 | 0 | 0 | 0 | 3.217 |

A texture analysis was undertaken. The analysis of sample firmness and toughness (as described in the specification hereinabove) revealed that the samples having an agar weight percent of 1.7% (1, 1c-1d, 1f) were relatively firm, and possessed an undesirable degree of toughness, rendering such formulations commercially undesirable as a gummy product.

The tackiness analysis showed that, for this limited run of samples, the inclusion of starch in these formulations provides, generally, lower tackiness, with the exception of one sample (1f). The relatively low tackiness in that sample (1f) was believed to be the result of the relatively high amount of agar in that formulation, which provided a sample that was relatively firm. Further, this sample, as noted above, would not be commercially acceptable, as it possessed excessive toughness.

Samples with and without starch were prepared from formulations of agar and LBG (1 v. 1c and 1a v. 1g), or a formulation of agar alone (1b v. 1h), with lower tackiness being observed for the samples containing starch. An exception was noted for samples prepared using a formulation containing a relatively high amount of agar (1.7 wt. %—1d v. 1f), wherein the inclusion of starch therein resulted in relatively higher tackiness. Although not desiring to be bound to any theory, this is believed to be due to a disrupted gel matrix possibly due to the use of the combination of gelling agents at relatively excessive levels, as well as excessive moisture pick up due to the absence of LBG.

A formulation with a weight percent of agar, LBG and starch in a relatively mid-range (1e—1.2, 0.1 and 4.9 wt. %, respectively) resulted in a tackiness of 1.722 g, which is higher relative to a formulation containing higher amounts of agar and starch (1—1.7, 0.1 and 9.7 wt. %, respectively), at 0.745 g, and lower relative to a formulation containing lower amounts of agar, LBG and no starch (1g—0.6, 0.1 and 0 wt. %, respectively), at 2.978 g.

### Example 2

The formulations described in Table 2, containing a mixture of multivitamins, were prepared, one with Ingredion Flojel® G (05060002) modified starch (Formula A in Table 2) and the other without starch (Formula B in Table 2).

The formulations were prepared by hydrating agar and LBG in water to form an initial mixture, and incorporating sugar, carbohydrate syrup and, in the case of Formulation A only, starch (dispersed in water) therein. The resulting mixture was then heated, with any heat-resistant vitamins and mineral being added to the mixture. Non-heat resistant ingredients may be added post cook. After the mixture was boiled, an aqueous acidic solution was added to lower the pH. Thereafter, the mixture was cooled to initiate formation of the gel structure, deposited into a plurality of molds, and allowed to fully cool, followed by a shakeout of the finished samples.

TABLE 2

| | Formula A | Formula B |
|---|---|---|
| | (weight percentage of | (weight percentage of |
| | ingredient in finished | ingredient in finished |
| Ingredient | product) | product) |
| Agar | 1.04 | 1.07 |
| Locust Bean Gum | 0.08 | 0.08 |
| Starch | 1.66 | — |
| Carbohydrate Syrup | 34.39 | 35.33 |
| Sugar | 33.22 | 34.14 |
| Vitamin Mixture | 15.20 | 15.21 |
| Citric Acid | 0.17 | 0.17 |
| Water | 14.15 | 13.91 |
| Polish | 0.10 | 0.10 |
| Total | 100.00 | 100.00 |

The vitamin mixture using in the examples consisted of: Vitamin A (3.26 wt. %, 2000 IU, in an acetate beadlet); Vitamin D3 (4.54 wt. %, 1000 IU), Vitamin E (32.42 wt. %, 40 IU), Riboflavin (Vitamin B2, 0.12 wt. %, 260 mcg), Niacin (Niacinamide, 1.22 wt. %, 3.2 mg), Pantothenic Acid (8.31 wt. %, 3 mg), Pyridoxine (Vitamin B6, 0.77 wt. %, 1.6 mg), Biotin (1.27 wt. %, 30 mcg), Folic acid (encapsulated, 1.04 wt. %, 160 mcg), Cyanocobalamin (Vitamin B12, 0.54 wt. %, 10 mcg), Ascorbic acid (Vitamin C, 28.92 wt. % 30 mg), Iodine (potassium iodide, 2.55 wt. %, 80 mcg), Manganese (manganese sulfate, 4.38 wt. %, 5 mg), zinc (zinc sulfate, 4.37 wt. %, 5 mg) and mannitol (10.20 wt. %).

An evaluation of the tackiness of 15 randomly selected samples from each formulation was conducted at two different time intervals: between about two and eight hours after shakeout and within the same day of production, and two days after shakeout, in the same manner, and using the same instrument, described in the specification hereinabove.

The samples with starch had a numerical average tackiness at 2-8 hours post-shakeout of 6.777 g (SD of 0.923) and an average tackiness of 6.631 g (SD of 1.295) two days later. The samples without starch had an average tackiness at shakeout of 10.273 g (SD of 2.072) and an average tackiness of 7.821 g (SD 3.078) two days later. Moreover, it was observed that moisture in the gummy samples without starch migrated to the surface, and in two days the samples began to migrate to the surface of each sample, with the samples sticking to one another, and exhibiting syneresis (although a reducing in tackiness over the two day post-shakeout period was observed). In comparison, the samples with starch did not stick to one another two days after shakeout, and thus remained free flowing and possessed relatively less surface moisture and tackiness.

These data demonstrate the advantages associated with the starch-containing formulation, as the relatively lower initial tackiness permits ease of processing and packaging of the gummy immediately after shakeout, as the samples exhibit relatively stable tackiness at between about 2-8 hours after shakeout (and within the same day of production) and two days thereafter, as well as minimal syneresis two days after shakeout, permitting the samples to forego sanding. A visual observation of the samples with and without starch were strikingly different, as all samples without starch exhibited adhesion to one another between 0-2 days, with the adhesion continuing to worsen over time.

### Example 3

Four formulations containing a mixture of multivitamins (the same mixture as described in Example 2) and calcium (as tricalcium phosphate), or magnesium citrate, were prepared as described in Table 3. Each formulation included agar and locust bean gum, with 2 of the 4 (Formulations C & D) also containing Ingredion Flojel® G (05060002) modified starch. The formulations were processed in the same manner described in Example 2 to provide gummy sample pieces.

An evaluation of the tackiness of 15 randomly selected samples from each formulation was conducted at two time intervals: between about 2-8 hours after shakeout (and within the same day of production), and two days after shakeout, using the same instrument and procedure described in the specification hereinabove.

TABLE 3

| | Formulation C | Formulation D | Formulation E | Formulation F |
|---|---|---|---|---|
| | (weight | (weight | (weight | (weight |
| | percentage of | percentage of | percentage of | percentage of |
| | ingredient in | ingredient in | ingredient in | ingredient in |
| Ingredient | finished product) | finished product) | finished product) | finished product) |
| Agar | 0.98 | 0.99 | 1.09 | 1.11 |
| Locust Bean | 0.07 | 0.07 | 0.04 | 0.04 |
| Gum | | | | |
| Starch | 0.86 | — | 1.02 | — |
| Carbohydrate | 35.05 | 35.53 | 33.84 | 34.49 |
| Syrup | | | | |
| Sugar | 31.33 | 31.80 | 24.90 | 25.38 |
| Magnesium | — | — | 32.99 | 33.00 |
| Citrate | | | | |
| Tricalcium | 11.45 | 11.45 | — | — |
| Phosphate | | | | |
| Vitamin Mixture | 6.13 | 6.06 | — | — |
| Citric Acid | 0.16 | 0.16 | 1.23 | 1.25 |
| Water | 13.88 | 13.84 | 4.78 | 4.63 |
| Polish | 0.10 | 0.10 | 0.10 | 0.10 |
| Total | 100.00 | 100.00 | 100.00 | 100.00 |

The tackiness of each formulation (an average obtained via an analysis of 15 randomly selected samples was assessed at two intervals: between about 2-8 hours after shakeout (and within the same day of production), and two days after shakeout, is set forth in Table 4.

TABLE 4

| | | | Average Tackiness | | | | | |
|---|---|---|---|---|---|---|---|---|
| | Average Tackiness | | (g) Two Days After | | Average Firmness | | Average Toughness | |
| Formulation | (g) at Shakeout | | Shakeout | | (g) | | (grams · sec) | |
| C | 6.986 | (SD 1.352) | 4.485 | (SD 0.929) | — | | — | |
| D | 7.04 | (SD 1.299) | 4.348 | (SD 1.096) | — | | — | |
| E | 6.559 | (SD 1.484) | 8.513 | (SD 1.666) | 822.294 | (SD 62.175) | 5077.09 | (SD 498.003) |
| F | 6.061 | (SD 1.584) | 6.481 | (SD 1.291) | 864.521 | (SD 68.509) | 4952.414 | (SD 466.3) |
| Commercially- | | | | | 3170.356 | (SD 422.467) | 24627.814 | (SD) 3470.505) |
| available | | | | | | | | |
| Magnesium | | | | | | | | |
| Supplement | | | | | | | | |
| Gummies | | | | | | | | |
| (83 mg | | | | | | | | |
| Magnesium as | | | | | | | | |
| Magnesium | | | | | | | | |
| Citrate/piece)* | | | | | | | | |

*This product also contains water, organic cane sugar, organic tapioca syrup, inulin, vegetable glycerin, agar, locust bean gum, natural flavors, citric acid, and organic black carrot (color).

The tackiness data obtained for formulations C and D described in Example 3 (vitamin mixture with calcium), and reported in Table 4, demonstrated no difference in samples that included starch relative to those that did not include starch. In contrast, the tackiness data for Formulation F (no starch, with magnesium) were similar (considering the SD) to Formulation F (with starch, with magnesium) at two days post-shakeout.

Firmness and toughness also were assessed in the manner described in the specification hereinabove, with values reported in Table 4 as numerical averages obtained via an analysis of 15 randomly selected samples. It was found that the magnesium-containing formulations E and F possessed significantly less toughness and firmness relative to a commercially-available magnesium-containing chewable gummy product. It is believed that the relatively lower toughness and firmness of formulations E and F will be more desirable from a consumer perspective.

Unless otherwise specified, all quantities described herein are by weight, and are based on the total weight of the finished formulation.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar references in the context of describing the invention (especially in the context of the following claims) are to be construed to cover both the singular and the plural, unless otherwise indicated herein or clearly contradicted by context. The use of the term “at least one” followed by a list of one or more items (for example; “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

Preferred embodiments of this invention are described herein. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

We claim:

1. A chewable oral formulation comprising about 0.5-2 wt. % agar, about 0.05-2 wt. % locust bean gum, about 0.1-2 wt. % starch, about 20-50 wt. % sugar, about 20-50 wt. % carbohydrate syrup, about 10-20 wt. % water and about 15 to about 40 wt. % of at least one vitamin, and optionally flavorants and/or colorants.

2. The chewable oral formulation according to claim 1, wherein the outer surface of the formulation is free of a water-absorbent excipient or desiccant.

3. The chewable oral formulation according to claim 2, wherein the formulation is free of gelatin, pectin, alginate, gellan, cellulose and carrageenan.

4. The chewable oral formulation according to claim 1, further comprising one or more of a flavorant and a colorant.

5. The chewable oral formulation according to claim 1, wherein the formulation has a water activity of less than about 0.75.

6. The chewable oral formulation according to claim 1, wherein the starch is a thin boiling starch or a natural starch.

7. The chewable oral formulation according to claim 6, wherein the Water Fluidity of the starch is from about 50-80.

8. The chewable oral formulation according to claim 7, wherein the Water Fluidity of the starch is from about 60-75.

9. The chewable oral formulation according to claim 1, wherein the formulation further comprises one or more of an antioxidant, folic acid, echinacea, garlic, a fructan, herb, or a herbal extract.

10. A chewable oral formulation comprising about 0.5-2 wt. % agar, about 0.01-2 wt. % locust bean gum, about 0.1-2 wt. % starch, about 20-50 wt. % sugar, about 20-50 wt. % carbohydrate syrup, about 10-20 wt. % water, about 5 to about 20 wt. % of at least one vitamin, about 10-30 wt. % calcium, and optionally flavorants and/or colorants.

11. The chewable oral formulation according to claim 10, wherein the formulation is free of gelatin, pectin, alginate, gellan, cellulose and carrageenan.

12. The chewable oral formulation according to claim 11, wherein the starch is a thin boiling starch or a natural starch.

13. The chewable oral formulation according to claim 12, wherein the amylose content of the starch ranges from about 5% to about 40%.

14. The chewable oral formulation according to claim 13, wherein the Water Fluidity of the starch is from about 50-80.

15. A chewable oral formulation comprising about 0.5-2 wt. % agar, about 0.005-0.1 wt. % locust bean gum, about 0.1-2 wt. % starch, about 20-30 wt. % sugar, about 30-40 wt. % carbohydrate syrup, about 1-10 wt. % water, and about 15-40 wt. % magnesium, and optionally flavorants and/or colorants.

16. The chewable oral formulation according to claim 15, wherein the formulation is free of gelatin, pectin, alginate, gellan, cellulose and carrageenan.

17. The chewable oral formulation according to claim 16, wherein the starch is a thin boiling starch or a natural starch.

18. The chewable oral formulation according to claim 17, wherein the Water Fluidity of the starch is from about 50-80.

19. The chewable oral formulation according to claim 15, wherein the outer surface of the formulation is free of a water-absorbent excipient or desiccant.

20. The chewable oral formulation according to claim 15, wherein the formulation has a water activity of less than about 0.75.

21. A package comprising a plurality of chewable oral formulations therein, wherein the chewable oral formulations comprise about 0.5-2 wt. % agar, about 0.005-0.1 wt. % locust bean gum, about 0.1-2 wt. % of a thin boiling starch or a natural starch having an amylose content ranging from about 5% to about 40% and a Water Fluidity range from about 50 to about 80 WF, about 20-30 wt. % sugar, about 30-40 wt. % carbohydrate syrup, about 1-10 wt. % water, and about 15-40 wt. % magnesium, and optionally flavorants and/or colorants, and wherein the plurality of chewable oral formulations are not sanded.
